(19) United States
(12) Patent Application Publication
(10) Pub. No.: US 2022/0105136 A1
(43) Pub. Date: Apr. 7, 2022

(54) COMPOSITION AND METHODS OF AN ADVANCED THERAPY FOR NEURODEGENERATIVE DISEASES

(71) Applicant: ASSOCIATION FOR THE ADVANCEMENT OF TISSUE ENGINEERING AND CELL BASED TECHNOLOGIES & THERAPIES A4TEC, Barco Guimarães, Braga (PT)

(72) Inventors: Helena Susana DA COSTA MACHADO FERREIRA, Joane (PT); Rui Luís GONÇALVES DOS REIS, Porto (PT); Nuno João MELEIRO ALVES DAS NEVES, Braga (PT)

(21) Appl. No.: 17/428,209

(22) PCT Filed: Feb. 19, 2020
(86) PCT No.: PCT/IB2020/051388

§ 371 Date: Aug. 3, 2021

(30) Foreign Application Priority Data
Feb. 20, 2019 (PT) 115322
Dec. 31, 2019 (EP) 19220277.8

(51) Int. Cl. A61K 35/28; A61K 47/36; A61K 9/127; A61K 47/22; A61K 47/24; A61K 38/18; A61K 9/06

(52) U.S. Cl. CPC A61K 35/28; A61K 47/36; A61K 9/127; A61K 9/06; A61K 47/24; A61K 38/185; A61K 47/22

(57) ABSTRACT

The present disclosure relates to a hydrogel comprising: hyaluronic acid, and liposomes physically crosslinked to hyaluronic acid, wherein said hydrogel encapsulates a cell type or a plurality of cell types. The hydrogel of the present disclosure shows a positive therapeutic effect in the treatment or therapy of multiple sclerosis. A pharmaceutical composition comprising a therapeutically effective amount of a hydrogel and a pharmaceutically acceptable excipient/carrier are also disclosed.

### TECHNICAL FIELD

The present disclosure relates to a hydrogel comprising: hyaluronic acid, and liposomes physically crosslinked to hyaluronic acid, wherein said hydrogel encapsulates a cell type or a plurality of cell types. The hydrogel of the present disclosure shows a positive therapeutic effect in the treatment or therapy of multiple sclerosis.

### BACKGROUND ART

Neurodegenerative diseases (NDs) are a heterogeneous group of diseases resulting from the progressive degeneration of the structure and function of particular subsets of neurons within the brain and/or spinal cord. Despite the selective neuronal loss, in the course of a disease there is frequently overlapping and convergence of the major NDs clinical manifestations, namely motor impairment, cognitive disability and/or dementia. Their prevalence and incidence are rising dramatically, and the World Health Organization predicts that by 2040 they will exceed cancer, becoming the second leading cause of death in the world after cardiovascular diseases. Indeed, NDs are one of the most important medical problems, being associated to a tremendous human suffering and socio-economic impact. Common NDs include Multiple Sclerosis (MS), Amyotrophic Lateral Sclerosis, Alzheimer's disease, Parkinson's disease and Huntington's disease.

Effective treatments for NDs do not exist, but stem cells have been shown a great potential for future ground-breaking research in developing innovative therapies. Indeed, stem cell therapy has potential to cure severe chronic conditions. For example, since 1950s that hematopoietic stem cell (HSC) transplantation is an established curative treatment for patients with advanced or refractory diseases. Besides HSC, there are several types of stem cells, such as neural (N)SC, embryonic (E)SC, mesenchymal (M)SC and induced pluripotent (iP)SC that can provide an unprecedented hope in NDs treatment. MSC present great advantages over other stem cells: i) easy collection, ii) high availability, iii) easy culture method, iv) low immunogenicity enabling allotransplantation, v) immunomodulatory properties, vi) no oncogenic transformation, and vii) no ethical concerns. Despite MSCs can be harvested from several sources, bone marrow-derived (B)MSCs are considered the best cell source as demonstrated by their use in different clinical trials or as reference. Besides stem cell type, formulation, time of administration and administration routes may also affect MSC therapy outcomes. Indeed, the cellular therapies outcomes can be improved by their engineering with natural-based biomaterials and/or through the combination with other adjunct therapies. MSC administration routes include systemic, intracerebral, intrathecal or intracerebroventricular (ICV) injection. Intrathecal and ICV injections are surely the most beneficial routes to treat NDs (intravenous injection has poor cell survival, high retention in the lungs, requires higher cell numbers and leads to poor cell survival and very impaired penetration into the central nervous system (CNS); and intracerebral transplantation can require multiple sites of transplantation due to MS multifocal nature). Additionally, the intrathecal and ICV routes of drug administration have been proven their safety and well-tolerance in a broad range of CNS diseases, even within the paediatric population. The cerebrospinal fluid (CSF) will allow the widespread distribution and integration of the cells at the multiple injured sites within the brain parenchyma and/or spinal cord.

The therapy outcomes can be further improved combining stem cells with other adjunct therapies. Indeed, due to the NDs multifactorial nature and complexity the “one target, one treatment” approach has been shifted into the “multi-target, multi-drug” model. However, this treatment modality also fails to inhibit the progression of the pathological process, to induce a protective mechanism and/or to regenerate the damaged tissues. Conversely, stem cells have the potential to address all the three pathways and consequently are more effective. However, the stem cells efficacy could be enhanced by including other therapeutic agents, such as growth factors, antibodies, genes or drugs into the liposomes.

Document US 2014/0105960 A1 discloses a hydrogel with encapsulated cells and liposomes, wherein the hydrogel can be a modified hyaluronic acid, covalently bonded. Nevertheless, the disclosed hydrogels are used to restore tissue function after an injury, but not as disease treatment, particularly NDs.

These facts are disclosed in order to illustrate the technical problem addressed by the present disclosure.

### General Description

Considering the recognized benefits and limitations of MSCs-based therapies, this disclosure proposes an ambitious breakthrough on the development and revolution of NDs treatment through a ground-breaking advanced therapy comprising BMSCs embedded in a hydrogel. This biocompatible and biodegradable hydrogel, physically crosslinked with liposomes, is only composed by biologically active compounds present in the CNS composition. In an embodiment, the hydrogel related to the present disclosure may be delivered directly into the ventricular space or spinal canal of the CNS. As CSF reaches all the brain and spinal cord, it will allow the widespread distribution of BMSCs at the multiple injured sites. The boosted bioavailability and the gradual and sustained release of the BMSCs into the CNS, by the time-controlled dissociation of the hydrogel, will underpin a prolonged and efficient protective and reparative effect of NDs and can allow decreasing the cells dose.

In an embodiment, afterwards the implantation of the hydrogel into the brain ventricular space or into the spinal canal, through a minimally invasive procedure, its efficacy can be monitored by magnetic resonance imaging (MRI) and quantified by relevant disease scores. The efficacy and safety of this new therapeutic approach will be first evaluated in established experimental models of MS. Indeed, MS is selected and utilized as a ‘proof of concept’ of the proposed technology, since it is the major cause of non-traumatic neurological disability in young adults.

The present disclosure relates to a hydrogel comprising: hyaluronic acid, and liposomes physically crosslinked to hyaluronic acid, wherein said hydrogel encapsulates a cell type or a plurality of cell types. The hydrogel of the present disclosure shows a positive therapeutic effect in the treatment or therapy of multiple sclerosis (MS).

The present disclosure relates to a hydrogel comprising liposomes and cells, in particular mesenchymal stem cells (MSCs), more in particular bone marrow-derived mesenchymal stem cells (B)MSCs.

The present disclosure also relates to a new therapy based in the delivery of stem cells embedded in a biocompatible and biodegradable hydrogel to be injected; in particular into the central nervous system (CNS), through an intrathecal or intracerebroventricular (ICV) injection.

The hydrogel of the present disclosure may be used in the treatment or therapy of a neurodegenerative disease (ND) that have an influence in the motor system, namely MS, amyotrophic lateral sclerosis, primary lateral sclerosis, Alzheimer's disease, Parkinson's disease, or Huntington's disease. The hydrogel of the present disclosure also enables delivering other therapeutic agents (e.g. genes, antibodies, growth factors and drugs).

MS is the main cause of chronic neurologic disability in young adults. Effective treatments for MS do not exist. MSC therapies are emerging as a promising alternative and with real curative potential. However, they still have limitations, such as poor cell survival. The present solution will develop and validate preclinically a ground-breaking advanced therapy comprising BMSCs embedded in a hydrogel. The biocompatible hydrogel, composed by biologically active compounds of the CNS composition, is physically crosslinked with liposomes that also enable the encapsulation of therapeutic agents. The advanced therapy can be delivered directly into the CNS through an intrathecal or an ICV injection. The boosted bioavailability and the gradual and sustained release of the BMSCs into the brain or spinal cord, by the time-controlled dissociation of the hydrogel, will underpin a prolonged and efficient protective and reparative effect of MS. A new treatment approach is proposed to overcome the unmet need of a safe and efficient therapy for this highly debilitating disease and related disorders.

Surprisingly, the experimental data of the present disclosure shows that the hydrogel of the present disclosure has a positive synergistic effect in the MS treatment.

In an embodiment for better results, the hydrogel may be used in medicine or veterinary. Namely, in the diagnostic, treatment or therapy of a ND. Preferably, for use in the diagnostic, treatment or therapy of a ND that have an influence in the motor system. More preferably, for use in the treatment or therapy of MS, or amyotrophic lateral sclerosis (ALS).

In an embodiment for better results, the amount of cells is at least 0.5×10⁶ cells, preferably at least 1×10⁶ cells, more preferably at least 5×10⁶ cells.

In an embodiment for better results, the hydrogel may comprise:

0.1-50% (wt/V_{hydrogel}) hyaluronic acid, preferably between 0.2-30% (wt/V_{hydrogel});

0.1 mM-1 M liposomes linked to hyaluronic acid, preferably <100 mM;

wherein said hydrogel encapsulates a cell type or a plurality of cell types.

In an embodiment for better results, the liposomes are crosslinkers of the hyaluronic acid. Also, the liposomes are physically crosslinked to hyaluronic acid by electrostatic interactions.

In an embodiment, the hyaluronic acid used to prepare the hydrogels related to the present disclosure has a molecular weight ranging from 0.7 kDa to 20,000 kDa; preferably 2 kDa to 2,000 kDa.

In a further embodiment, the hydrogel viscosity at 25° C. ranges from 1 to 10,000 Pa·s; preferably 3 to 1000 Pa·s.

In an embodiment for better results, the cell type or a plurality of cell types are stem cells, endothelial cells, endothelial progenitor cells, hematopoietic progenitor cells, hematopoietic stem cells, neural progenitor cells, neural stem cells, induced pluripotent stem cells, amniotic fluid stem cells, amniotic membrane stem cells, umbilical cord stem cells, genetically engineered cells, MSC, or combinations thereof. Preferably, the cell type or plurality of cell types are adult MSCs. More preferably, the adult MSCs are BMSCs.

In an embodiment for better results, the liposomes are unilamellar liposomes (LUVs) prepared, for instance, by the ethanol injection or the thin film hydration method followed by extrusion, ultrasound and high-pressure homogenization.

In an embodiment for better results, the liposomes are LUVs prepared by the thin film hydration method followed by extrusion.

In an embodiment for better results, the liposome is an anionic liposome.

In an embodiment for better results, the liposome comprises vitamin E and phospholipids and fatty acids.

In an embodiment for better results, the liposomes comprise at least two phospholipids.

In an embodiment for better results, the fatty acids comprise omega-3.

In an embodiment for better results, the liposomes further encapsulate a therapeutic agent. Preferably the therapeutic agent is a growth factor, antibody, genetic material, anti-inflammatory, antibiotic, antipyretic, analgesic, anticancer, or mixtures thereof. More preferably, the grow factor is a neuroprotective or a neurotrophic growth factor.

In an embodiment for better results, the hydrogel is injectable, more in particular an intrathecal or ICV injection.

Another aspect of the present composition relates to a pharmaceutical composition comprising a therapeutically effective amount of a hydrogel described in the present disclosure and a pharmaceutically acceptable excipient/carrier.

In an embodiment, the present disclosure relates to:
(a) design of a new hydrogel used as the cell carrier,
(b) ventricular and intrathecal delivery, as the common administration route of cells, in particular MSCs and contrast agents is the intravenous route;
(c) the hydrogel of the present disclosure allows the in situ gradual and sustained MSCs release,
(d) the hydrogel of the present disclosure increases stem cells efficacy due to their prolonged residence time into the CNS,
(e) the administration routes allow easy on-site collection of CSF samples or injection of contrast agents,
(f) the hydrogel of the present disclosure allows using non-invasive imaging techniques for MSCs tracking and disease monitoring.

In an embodiment, the safety and efficacy of the new therapeutic approach will be evaluated in established experimental autoimmune encephalomyelitis (EAE) animal models, due to their similarities with the most common human inflammatory demyelinating disease, namely MS. As MS, EAE is also a complex condition and properly mimics the key pathological features of MS: inflammation, demyelination, axonal loss or damage and gliosis. Consequently, EAE is widely used to identify the pathological mechanisms of MS and to investigate new therapeutic agents.

### BRIEF DESCRIPTION OF THE DRAWINGS

The following figures provide preferred embodiments for illustrating the disclosure and should not be seen as limiting the scope of invention.

FIG. 1: Biological performance of the BMSCs in the presence of different concentrations of liposomes (7.5 and 15 mM) and HA (0.25 and 0.5% (Wt/V_{hydrogel})): A) cell viability, B) cell proliferation and C) total protein synthesis after 1, 3 and 7 days of culture. Asterisk (*) denotes significant differences (p<0.01) compared to the control (Ctr).

FIG. 2: Confocal microscopy images of the hydrogel composed by HA linked to fluorescein and liposomes labelled with Dy-750 (as light grey). Scale bar, 100 μm.

FIG. 3: DSC thermograms of LUVs, HA and liposomes-in-HA hydrogel presenting different concentrations of lipid (7.5 and 15 mM). For clarity, along Y-axis, the signals are shifted.

FIG. 4: Rheological behaviour of 1% (wt/V_{hydrogel}) HA-based hydrogels crosslinked or not with 7.5 and 15 mM liposomes, at 25 and 37° C. A) Shear elastic modulus (G′, Pa) and B) shear viscous modulus (G″, Pa) measurements as a function of frequency (s⁻¹). C) Shear viscosity (Pa·s) measurement as a function of shear rate (s⁻¹).

FIG. 5: Cross-sectional SEM pictures of hydrogels composed by 1% (wt/V_{hydrogel}) HA (a) or by 1% (wt/V_{hydrogel}) HA and 7.5 mM (b) or 15 mM (c) of liposomes. Magnification 500×; scale bar 50 μm.

FIG. 6: Confocal microscopy images of Live/Dead staining with Calcein AM/PI (light grey: live cell) of BMSCs cultured in tissue culture polystyrene (TCPS) coverslips (a) or in hydrogels composed with 1% (wt/V_{hydrogel}) HA and 7.5 (b) or 15 (c) mM of liposomes, after 7 days of culture.

FIG. 7: A—Coronal section of the rat brain (adapted from G. Paxinos, C. Watson, The rat brain in stereotaxic coordinates, Elsevier Inc. 2007): CC—corpus callosum; Cg1—cingulate cortex, area 1; Cg2—cingulate cortex, area 2; chp—choroid plexus; CPu caudate putamen (striatum); GP globus pallidus; LSD—lateral septal nu, dorsal part; LV—lateral ventricle; 3V—3rd ventricle. Fluorescence microscopy images of nucleic staining with DAPI (light grey, B) and liposomes-in-HA hydrogel (light grey, C) obtained from rat brain after 1 week of ICV administration. Magnification 40×.

FIG. 8: Average of the speed and total distance covered in 5 min evaluated using open field activity.

FIG. 9: Evaluation of cumulative disease disability after ICV injection of the hydrogel incorporating or not BMSCs (A) and of the cells suspension (B) in EAE rats.

### DETAILED DESCRIPTION

The present disclosure relates to a hydrogel comprising liposomes and cells, in particular adult stem cells, more in particular bone marrow-derived mesenchymal stem cells (BMSCs).

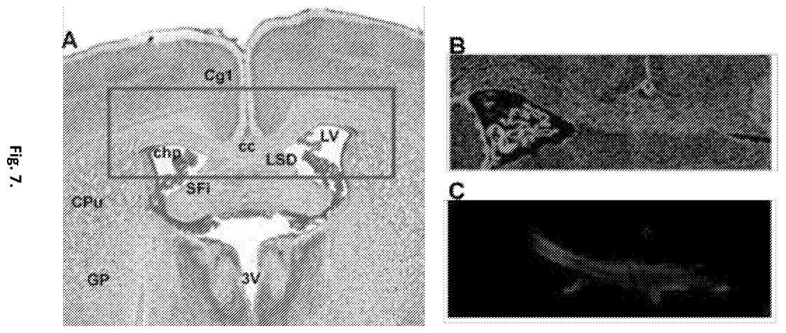

The present disclosure also relates to a new therapy based in the delivery of stem cells embedded in a biocompatible and biodegradable hydrogel to be injected; in particular, into the central nervous system (CNS), through an intrathecal or intracerebroventricular (ICV) injection (see FIG. 7).

In FIG. 7 it is possible to observe that after ICV administration, the hydrogel descended and diffused to both sides of the ventricle into the corpus callosum. This white matter structure, is one of the sites more affected, even in early phases of NDs (e.g. MS and Alzheimer's disease) and allows the interhemispheric communication. Consequently, its damage is considered to be responsible for important deficits in the integration of sensory, motor and cognitive pathways. Therefore, the hydrogel distribution pattern of the hydrogel in this bundle of nerve tissue further supports the use of this innovative therapy in NDs treatment.

The present disclosure relates to a therapy based in the delivery of cells, in particular stem cells, more in particular BMSCs embedded in a hyaluronic acid hydrogel comprising liposomes, to be injected into the CNS, through an intrathecal or ICV injection. The hydrogel also enables delivering other therapeutic agents (e.g. genes, growth factors, antibodies and drugs).

In an embodiment, the present disclosure relates to: a) provide a formulation of BMSCs encapsulated into a hydrogel comprising components naturally present in CNS and in clinical use; b) validate the therapeutic potential of the proposed therapy after its injection into the brain ventricular space or spinal canal through a minimally invasive procedure; c) exceed the performance of the currently used MSC-based treatments; d) increase future treatment modalities, by including therapeutic agents into the liposomes.

In an embodiment, the disclosed treatment is easily injected, safe, effective and well tolerated. Hydrogels are often used as pharmaceutical formulations, due to their suitable characteristics for mild immobilization of cells/therapeutic agents and injection by different administration routes. Among the existing biomaterials, hyaluronic acid (HA) presents critical physicochemical and biological properties that make it the ideal candidate for the interaction with human stem cells and development of carriers to treat CNS disorders. Indeed, HA is one of the main structural components of the brain extracellular matrix. High molecular weight HA chemically unmodified is used. The disclosed treatment: a) promotes the use of non-toxic, environmentally friendly solvents for manufacturing, b) avoids the production of small molecular weight fragments that can be pro-inflammatory and c) maintains the biological and anti-inflammatory properties of HA. The suitable rheological properties of the HA hydrogel are tailored with liposomes. Liposomes being composed by lipids, that comprise 60% of brain matter, are totally biodegradable, safe and well tolerated according to the clinically approved liposome-based therapies. Liposomes are composed of vitamin E (antioxidant) and two phospholipids, namely phosphatidylcholine (PC) and phosphatidylserine (PS) that are common components of brain supplements. Indeed, PC and PS are fundamental to support structural, biochemical and cell signalling functions in brain. Additionally, omega-3 fatty acids can be incorporated.

In an embodiment, pre-cultured and expanded BMSCs are incorporated into the hydrogel carrier. BMSCs have several features relevant to NDs treatment (cease pathological processes, can enhance protective mechanisms and regenerate damaged tissues) and in particular for MS (e.g. re-myelination, neuroprotection, and reduced gliosis). The in situ sustained release of cells heightens their number and allows a prolonged and efficient treatment of the injury sites in the brain and/or spinal cord. This surprisingly leads to a lower dose of BMSCs in one or multiple administrations. To obtain an efficient release of cells from the hydrogel, their appropriate residence time, integrity and controlled release is disclosed. For that, HA and liposomes concentration as well as the BMSC ratio are judiciously adjusted. Live Cell Imaging performs the in vitro monitoring. For in vivo studies, magnetically or green fluorescent protein labelled cells and MRI or fluorescence molecular tomography with micro computed tomography (μCT) are used. If the BMSCs release is not as desired, the preparation of the hydrogel can be/is reassessed. The potential of cells differentiation pre- and post-transplantation is evaluated by morphological and immunocytochemical methods.

In an embodiment, the safety and efficacy of the new advanced therapy is firstly evaluated in established Lewis and Dark Agouti strains of experimental autoimmune encephalomyelitis (EAE) rats. Indeed, MS is selected and utilized as a ‘proof of concept’ of the advanced therapy to have in the future a higher evidence of its efficacy in increase patient's health and lifetime. Indeed, contrariwise to the generality of NDs (with prevalence and incidence usually rising dramatically with age) MS is the major cause of non-traumatic neurological disability in young adults.

In an embodiment, the use of EAE rat models instead of mouse models, presents several advantages. EAE rat models presenting different clinical courses, namely a relapsing-remitting model and a secondary progressive MS model are used. After the advanced therapy injection, animals' daily evaluation of the clinical score and neuropathology levels are performed. MRI is also used to evaluate the disease state of EAE after delivery of clinically approved gadolinium-based contrast agents. The analysis of the biomedical images surprisingly shows a better monitoring of the disease state and the quantitative follow-up of the advanced therapy efficacy. One or more injections of the advanced therapy, contrast agents injection or CSF sampling, when necessary can be performed (as the Ommaya reservoirs in clinical use).

In an embodiment, therapeutic agents are released from the liposomes.

The present disclosure relates to an advanced therapy consisting in BMSCs embedded in a biocompatible and biodegradable hydrogel to be injected either into the brain ventricular space or spinal canal through a minimally invasive procedure.

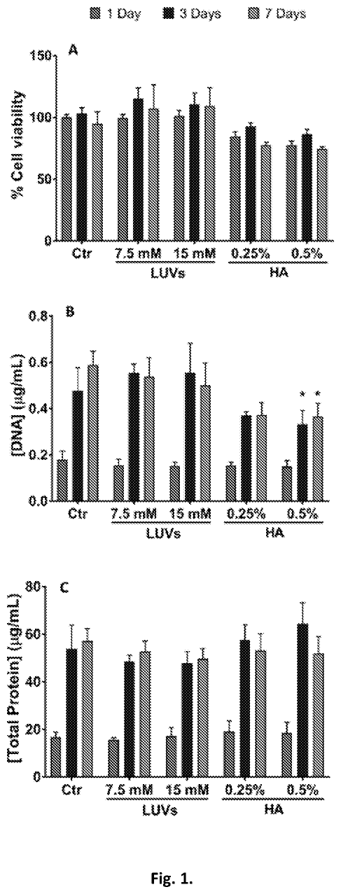

In an embodiment, the cytotoxicity of the hydrogel components was assessed (FIG. 1). As can be seen in FIG. 1 both liposomes and HA are cytocompatible with BMSCs.

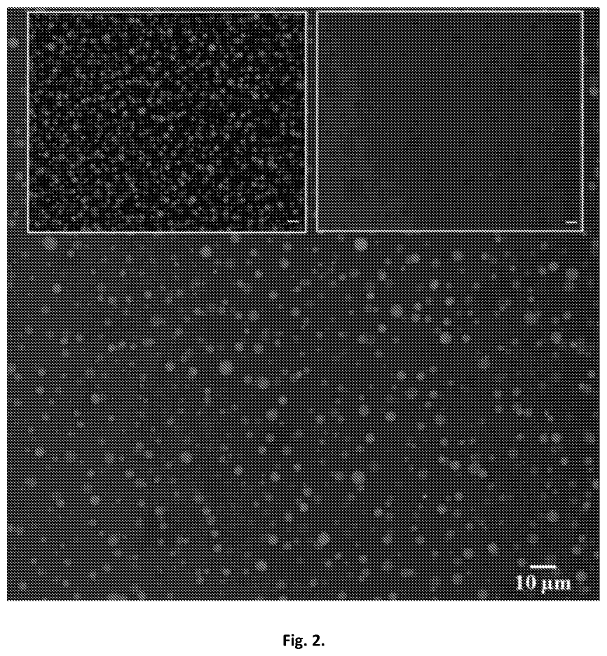

In an embodiment, HA and liposomes are mixed to form the hydrogel. For the hydrogel preparation, a mixture of HA and liposome's suspension is magnetically stirred until complete dissolution of the polymer and homogeneous distribution of the liposomes. In an embodiment a laser scanning confocal microscopy imaging system is used to visualize and localize the liposomes in the HA hydrogel. In FIG. 2 it is possible to visualize the uniform distribution of liposomes (light grey) in the HA matrix (dark background).

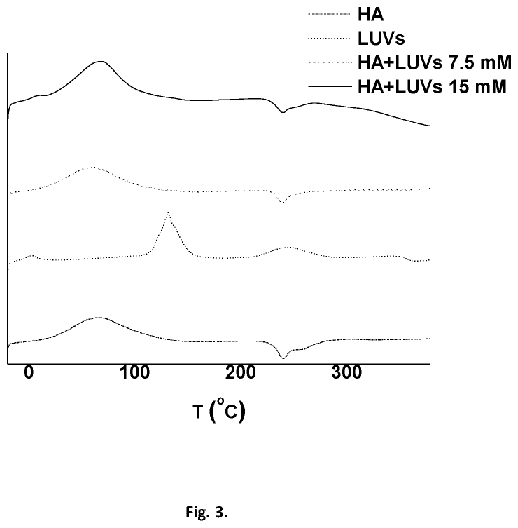

In an embodiment, the mixture is thermally stable (FIG. 3).

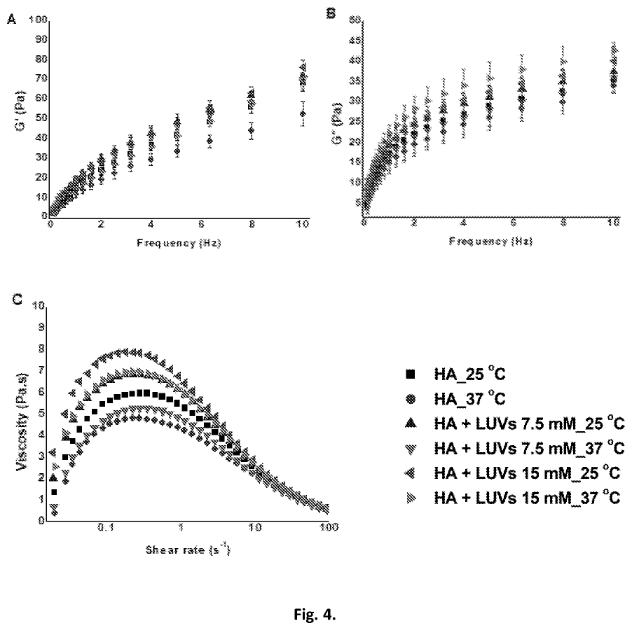

In an embodiment, the assessment of the hydrogel rheological behavior demonstrated that the compounding with liposomes increased the viscous and the elastic modulus of the HA matrix, as well as, the viscosity of the formulation (FIG. 4). In the state of the art, the assessment of the hydrogel rheological properties may be performed by many methods. In the present disclosure, the rheological behavior of the hydrogel presenting or not liposomes into its composition was determined using a Kinexus Prot Rheometer (Malvern). The viscosity and viscoelasticity assessment of the hydrogels was performed at 25 and 37° C., particularly at 37° C., with a plate geometry of 20 mm in diameter. The viscosity was determined using a shear rate from 0.01 to 1000 s⁻¹ and the viscoelastic properties were investigated using a frequency range from 0.01 to 10 Hz at 0.2% shear strain (calculated from amplitude sweep with linear viscoelastic region—LVER—determination measurements).

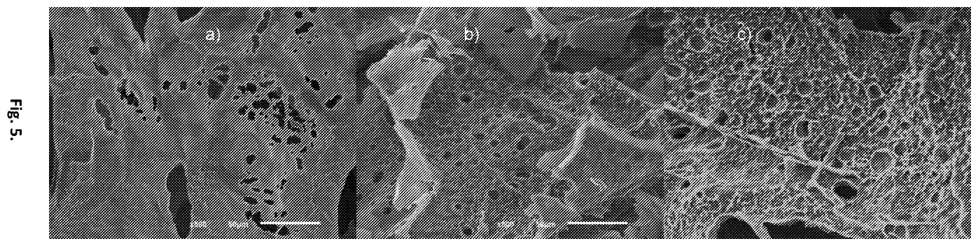

In an embodiment, the morphology of the liposomes-in-HA hydrogels was also probed by scanning electron microscopy (SEM). Representative images of the cross sections are depicted in FIG. 5. HA hydrogels containing liposomes have a more irregular surface.

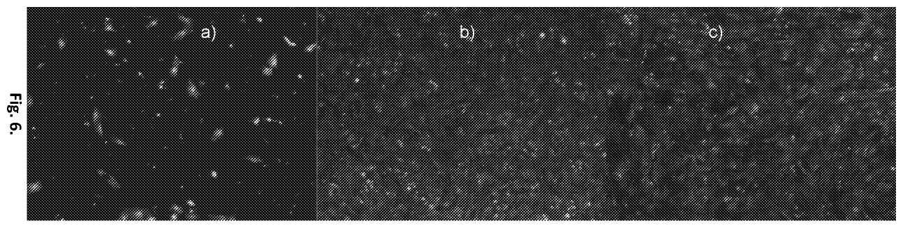

In an embodiment, the 3D encapsulation of cells demonstrated that BMSCs were able to adhere to, to survive and to proliferate within hydrogels in a higher extension than in 2D culture. In FIG. 6 it is possible to observe that the cells in the hydrogels are viable (grey), whereas some cell death is observed in tissue culture polystyrene controls.

In an embodiment, a preliminary assay to determine the residence time, integrity and controlled release of cells from the hydrogel was monitored by Live Cell Imaging. This technique allowed observing that BMSCs were able to adhere on cell plate and maintain their integrity until 48 h of microscopy observation.

In an embodiment, BMSCs were characterized by specific cell-surface markers. The cells were positive for CD90, CD44 and CD106 and negative for hematopoietic markers CD45 and CD34, confirming the identity of isolated BMSCs.

In an embodiment, in vivo safety of the developed hydrogel, after brain ventricular delivery in rats, was also confirmed until 2 weeks. An example of the obtained histology images is shown in FIG. 7. As can be observed the hydrogel after administration diffuses into the corpus callosum, which allows the interhemispheric communication, taking part in most of the cognitive pathways. Moreover, being corpus callosum one of the sites more affected, even in a very early phase of MS, the predisposition of the hydrogel for this bundle of nerve tissue can be ideal to treat MS patients.

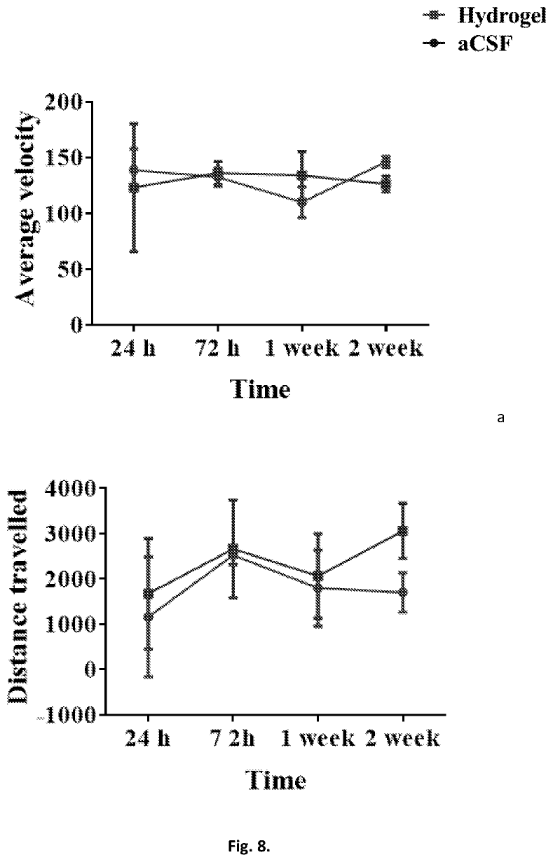

In an embodiment, the open field activity monitoring system showed that the rats treated with the hydrogel as well as the controls (injection of artificial CSF) had a similar locomotor and behavioural activity. FIG. 8 presents two parameters evaluated in the test of locomotion. This Figure shows that there are no statistical differences between the 2 groups of rats in terms of the average velocity. Surprisingly these results show that the administration of the hydrogel led rats to travel a higher distance after 2 weeks post injection. These data shows that the hydrogel itself, due to its valuable composition, has a positive synergistic effect in the MS treatment.

In an embodiment, the therapy is initiated only after individuals develop clinical symptoms of MS, and the advanced therapy is administered at day 10 after EAE induction.

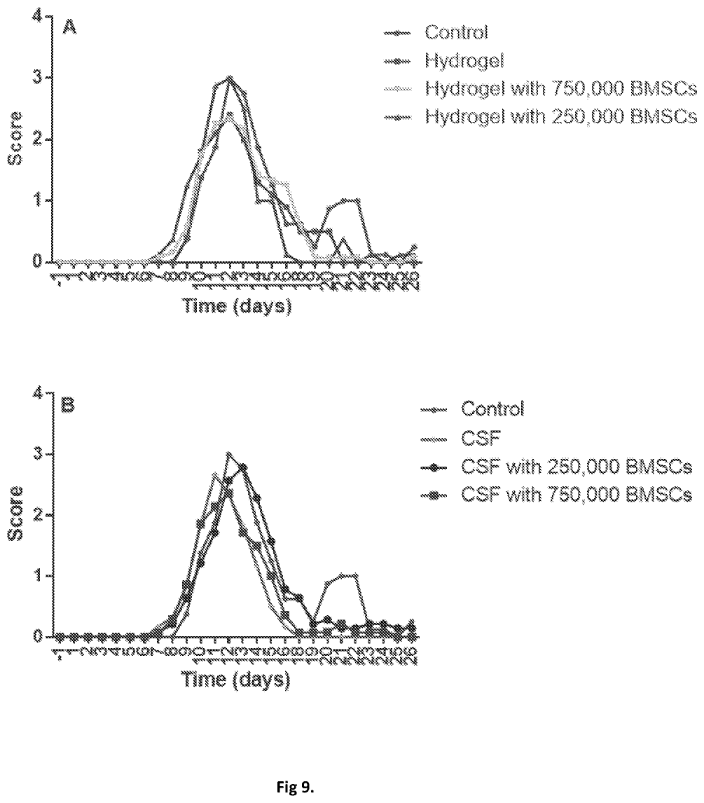

In an embodiment, EAE is scored on scale 0 (no obvious changes in motor function compared to non-immunized rats) to 5 (rat is found dead due to paralysis). From FIG. 9 it is possible to observe that the hydrogel incorporating 750,000 cells, a significantly lower number of cells than is usually reported in the literature (1×10⁶), caused a significant decrease in the maximum mean clinical score and the average mean clinical score, compared with the EAE group, and eliminated the relapse.

In conclusion, these results illustrate that this is the effective way to reduce patients' disability-adjusted life years (loss of years expected to be lived in full health as a result of MS).

Where singular forms of elements or features are used in the specification of the claims, the plural form is also included, and vice versa, if not specifically excluded. For example, the term “a polysaccharide” or “the polysaccharide” also includes the plural forms “polysaccharides” or “the polysaccharides,” and vice versa. In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skilled in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following claims further set out particular embodiments of the disclosure.
[1] T. A. Yacoubian, in Drug Discovery Approaches for the Treatment of Neurodegenerative Disorders, Academic Press, 2017, DOI: https://doi.org/10.1016/8978-0-12-802810-0.00001-5, pp. 1-16.
[2] G. G. Kovacs, EMJ Neurol., 2014, 1, 78-86.
[3] M. Freitas-Andrade and C. C. Naus, Neuroscience, 2016, 323, 207-221.
[4] M. K. Juric, S. Ghimire, J. Ogonek, E. M. Weissinger, E. Holler, J. J. van Rood, M. Oudshoorn, A. Dickinson and H. T. Greinix, Front Immunol., 2016, 7, 470.
[5] M. Boo, S. M. van Walraven, J. Chapman, B. Lindberg, A. H. Schmidt, B. E. Shaw, G. E. Switzer, E. Yang and T. Egeland, Blood, 2011, 117, 21-25.
[6] A. Torsvik, G. V. Røsland, A. Svendsen, A. Molven, H. Immervoll, E. McCormack, P. E. Lønning, M. Primon, E. Sobala, J.-C. Tonn, R. Goldbrunner, C. Schichor, J. Mysliwietz, T. T. Lah, H. Motaln, S. Knappskog and R. Bjerkvig, Cancer Res., 2010, 70, 6393-6396.
[7] I. Ullah, Raghavendra B. Subbarao and Gyu J. Rho, Biosci Rep., 2015, 35, e00191.
[8] A. Uccelli, A. Laroni and M. S. Freedman, Lancet Neurol., 2011, 10, 649-656.
[9] I. De La Pena, M. Pabon, S. Acosta, P. R. Sanberg, N. Tajiri, Y. Kaneko and C. V. Borlongan, Cell Med., 2014, 6, 123-127.
[10] W. Baek, Y. S. Kim, S. H. Koh, S. W. Lim, H. Y. Kim, H. J. Yi and H. Kim, J Neurosurg. Sci., 2012, 56, 261-263.
[11] J. L. Cohen-Pfeffer, S. Gururangan, T. Lester, D. A. Lim, A. J. Shaywitz, M. Westphal and I. Slavc, Pediatr Neurol., 2017, 67, 23-35.
[12] W. J. Brownlee, T. A. Hardy, F. Fazekas and D. H. Miller, Lancet, 2017, 389, 1336-1346.
[13] S. Amorim, A. Martins, N. M. Neves, R. L. Reis and R. A. Pires, J. Mater. Chem. B, 2014, 2, 6939-6946.
[14] A. Bignami, M. Hosley and D. Dahl, Anat Embryol, 1993, 188, 419-433.
[15] S. Hashioka, Y. H. Han, S. Fujii, T. Kato, A. Monji, H. Utsumi, M. Sawada, H. Nakanishi and S. Kanba, Free Radic Biol Med., 2007, 42, 945-954.
[16] J. F. J. Bogie, P. Stinissen and J. J. A. Hendriks, Acta Neuropathol., 2014, 128, 191-213.
[17] G. Dvoriantchikova, C. Agudelo, E. Hernandez, V. I. Shestopalov and D. Ivanov, J Cereb Blood Flow Metab., 2009, 29, 1755-1759.
[18] H.-Y. Kim, B. X. Huang and A. A. Spector, Prog Lipid Res., 2014, 56, 1-18.
[19] P. H. Lee and H. J. Park, J Clin Neurol., 2009, 5, 1-10.
[20] A. Wilkins, K. Kemp, M. Ginty, K. Hares, E. Mallam and N. Scolding, Stem Cell Res., 2009, 3, 63-70.
[21] E. Wallström and T. Olsson, in Sourcebook of Models for Biomedical Research, ed. P. M. Conn, Humana Press, Totowa, N.J., 2008, DOI: 10.1007/978-1-59745-285-4_56, pp. 547-556.
[22] A. Ozturk, S. A. Smith, E. M. Gordon-Lipkin, D. M. Harrison, N. Shiee, D. L. Pham, B. S. Caffo, P. A. Calabresi and D. S. Reich, Mult Scler., 2010, 16, 166.

1. A hydrogel comprising:
hyaluronic acid, and
liposomes physically crosslinked to hyaluronic acid,
wherein said hydrogel encapsulates a type of cell or a plurality of types of cells.

2. (canceled)

3. (canceled)

4. A method for using the hydrogel according to claim 1, in the treatment or therapy of multiple sclerosis or amyotrophic lateral sclerosis.

5. The hydrogel according to claim 1, wherein the liposomes are physically crosslinked to hyaluronic acid by electrostatic interactions.

6. The hydrogel according to claim 1, wherein the liposome is an anionic liposome.

7. The hydrogel according to claim 1, wherein the liposomes comprise vitamin E, phospholipid and fatty acid.

8. The hydrogel according to any claim 1, wherein the liposomes comprise at least two phospholipids.

9. The hydrogel according to claim 7, wherein the fatty acid comprises omega-3.

10. The hydrogel according to claim 1, wherein the molecular weight of hyaluronic acid ranges from 0.7 kDa to 20,000 kDa.

11. The hydrogel according to claim 1, wherein the hydrogel viscosity at 37° C. ranges from 1 to 10,000 Pa·s.

12. The hydrogel according to claim 1, wherein the amount of cells is at least 0.5×10⁶ cells.

13. The hydrogel according to claim 1, wherein there is:
0.1-50% (wt/V_{hydrogel}) hyaluronic acid; and
0.1 mM-1 M liposomes linked to hyaluronic acid, preferably <100 mM;
wherein said hydrogel encapsulates a cell type or a plurality of cell types.

14. The hydrogel according to claim 1, wherein the liposomes crosslink the hyaluronic acid.

15. The hydrogel according to claim 1, wherein the type of cell or a plurality of cell types are selected from the group consisting of: stem cells, endothelial cells, endothelial progenitor cells, hematopoietic progenitor cells, hematopoietic stem cells, neural progenitor cells, neural stem cells, induced pluripotent stem cells, amniotic fluid stem cells, amniotic membrane stem cells, umbilical cord stem cells, genetically engineered cells, mesenchymal stem cell, and combinations thereof.

16. The hydrogel according to claim 1, wherein the type of cells or the plurality of types of cells are adult mesenchymal stem cells.

17. The hydrogel according to claim 16, wherein adult mesenchymal cells are bone marrow-derived mesenchymal stem cells.

18. The hydrogel according to claim 1, any of the previous claims wherein the liposomes further encapsulate a therapeutic agent or a combination of therapeutic agents.

19. The hydrogel according to claim 18, wherein therapeutic agent is selected from the group consisting of: a growth factor, antibody, genetic material, anti-inflammatory, antibiotic, antipyretic, analgesic, anticancer, and mixtures thereof.

20. The hydrogel according to claim 19, wherein the growth factor is a neuroprotective or a neurotrophic growth factor.

21. The hydrogel according to claim 1, wherein the hydrogel is injectable.

22. A pharmaceutical composition comprising a therapeutically effective amount of the hydrogel recited in claim 1 and a pharmaceutically acceptable excipient/carrier.
